(19) United States
(12) Patent Application Publication
(10) Pub. No.: US 2023/0131209 A1
(43) Pub. Date: Apr. 27, 2023

(54) TREATMENT DEVICE AND ENDOSCOPE SYSTEM

(71) Applicant: OLYMPUS MEDICAL SYSTEMS CORP., Tokyo (JP)

(72) Inventor: Hiroaki KAGAWA, Sagamihara-shi (JP)

(73) Assignee: OLYMPUS MEDICAL SYSTEMS CORP., Tokyo (JP)

(21) Appl. No.: 17/971,389

(22) Filed: Oct. 21, 2022

Related U.S. Application Data
(60) Provisional application No. 63/270,593, filed on Oct. 22, 2021.

(51) Int. Cl. A61B 1/313; A61B 1/05; A61B 17/29; A61B 1/00

(52) U.S. Cl. CPC A61B 1/313; A61B 1/05; A61B 17/29; A61B 1/00193; A61B 2017/2908; A61B 2017/2947; A61B 2017/2929; A61B 2017/00738

(57) ABSTRACT

A treatment device includes a shaft extending along a longitudinal axis and including a plurality of segments arranged along the longitudinal axis, a treatment portion at a distal-end side of the shaft, an operation portion at a proximal-end side of the shaft, and an imaging device. A first segment of the plurality of segments is at the distal-end side of a second segment of the plurality of segments and is rotatably attached to the second segment at a first pivot point. The imaging device is provided in the second segment. The first segment is rotatable about the first pivot point relative to the second segment such that a longitudinal axis of the first segment intersects a longitudinal axis of the second segment at a first angle and at least a part of the treatment portion enters a field of view of the imaging device.

### RELATED APPLICATION DATA

This application is based on and claims priority under 37 U.S.C. § 119 to U.S. Provisional Application No. 63/270,593 filed on Oct. 22, 2021, the entire contents of which are incorporated herein by reference.

### FIELD OF THE DISCLOSURE

The present disclosure relates to a treatment device and an endoscope system.

### BACKGROUND

Conventionally, in a laparoscopic surgery, a technique is used to insert a treatment device, an endoscope, and the like through different holes (openings) opened in the abdominal wall to perform a treatment. The treatment device and the endoscope are introduced into the abdominal cavity through different trocars from each other. Accordingly, there is a case in which a view direction of the endoscope is not coincided with an insertion direction of the treatment device such that the surgeon cannot intuitively operate the treatment device based on the captured image of the endoscope.

Also, in an endoscopic surgery using a flexible endoscope, there is a case in which the view direction of the endoscope is not coincided with the insertion direction of the treatment device such that the surgeon cannot intuitively operate the treatment device.

A surgical treatment device described in Patent Document 1 is configured to operate the treatment device and the imaging device to be moveable to a position offset from an axis such that it is easy to image the range treated by the treatment device by the imaging device.
Patent Document 1: Japanese Unexamined Patent Application, First Publication No. 2008-155030

### SUMMARY

According to a first aspect of the present disclosure, a treatment device includes a shaft extending along a longitudinal axis and including a plurality of segments arranged along the longitudinal axis, a treatment portion at a distal-end side of the shaft, an operation portion at a proximal-end side of the shaft and configured to operate the treatment portion, and an imaging device. A first segment of the plurality of segments is adjacent to a second segment of the plurality of segments. Along the longitudinal axis of the shaft, the first segment is at the distal-end side of the second segment. The imaging device is provided in the second segment. The first segment is rotatably attached to the second segment at a first pivot point. The first segment is rotatable about the first pivot point relative to the second segment such that a longitudinal axis of the first segment intersects a longitudinal axis of the second segment at a first angle that is less than 180 degrees and at least a part of the treatment portion enters a field of view of the imaging device.

An endoscope system according to a second aspect of the present disclosure includes the above-described treatment device; an endoscope; and a processor configured to process an image captured by the imaging portion of the treatment device and an image captured by the endoscope.

According to a third aspect of the present disclosure, a treatment device includes a first shaft portion extending along a longitudinal axis, a second shaft portion rotatably attached to the first shaft portion, a treatment portion provided at a distal end of the second shaft portion, an operation portion provided at a proximal end of the first shaft portion and configured to operate the treatment portion, and an imaging device provided on the first shaft portion such that at least part of the treatment portion enters a field of view of the imaging device when the second shaft portion is rotated. The second shaft portion configured to rotate in a direction intersecting with the longitudinal axis.

The treatment device and the endoscope system according to the present disclosure facilitate the imaging of the range treated by the treatment device with the imaging device.

### BRIEF DESCRIPTION OF THE DRAWINGS

FIG. 1 is a view showing an overall configuration of an endoscope system including a treatment device according to a first embodiment.

FIG. 2 is a view showing an insertion portion of the treatment device.

FIG. 3 is a view transparently showing part of a shaft when rotation mechanism of the treatment device is rotating.

FIG. 4 is a view showing an operation of the treatment device.

FIG. 5 is a view showing an insertion portion of a treatment device according to a second embodiment.

FIG. 6 is a view showing an insertion portion of a treatment device according to a third embodiment.

FIG. 7 is a view showing a shaft when rotation mechanism of the treatment device is rotating.

FIG. 8 is a view showing part of an endoscope system according to a fourth embodiment.

### DETAILED DESCRIPTION

### First Embodiment

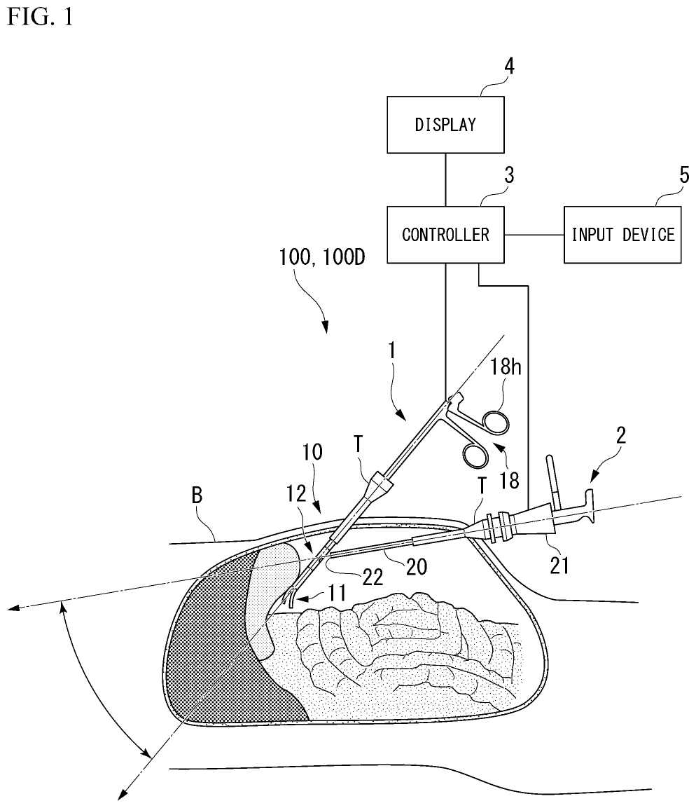

A first embodiment of the present disclosure will be described with reference to FIG. 1 to FIG. 4. FIG. 1 is a view showing the overall configuration of an endoscope system 100 including a treatment device 1 according to the present embodiment.

### [Endoscope System 100]

The endoscope system 100 includes a treatment device 1, an endoscope 2, a control device 3, a display device 4, and an input device 5, as shown in FIG. 1. The endoscope system 100 is a system configured for performing procedures in a laparoscopic surgery by inserting the treatment device 1, the endoscope 2, and the like through different holes (openings) opened in the abdominal wall.

### [Treatment Device 1]

The treatment device 1 has an elongated insertion portion 10 that is insertable into the patient's abdominal cavity, and an operation portion 18 provided on the proximal end side of the insertion portion 10. The insertion portion 10 is introduced into the abdominal cavity through a trocar T punctured in the abdomen B of the patient. The operation portion can be any suitable structure that can be manipulated by an operator, such as a medical professional, to operate the treatment portion to affect a procedure on a patient. Example operation portions include a handle, a knob, a joystick, and variations, substitutions and equivalents thereof.

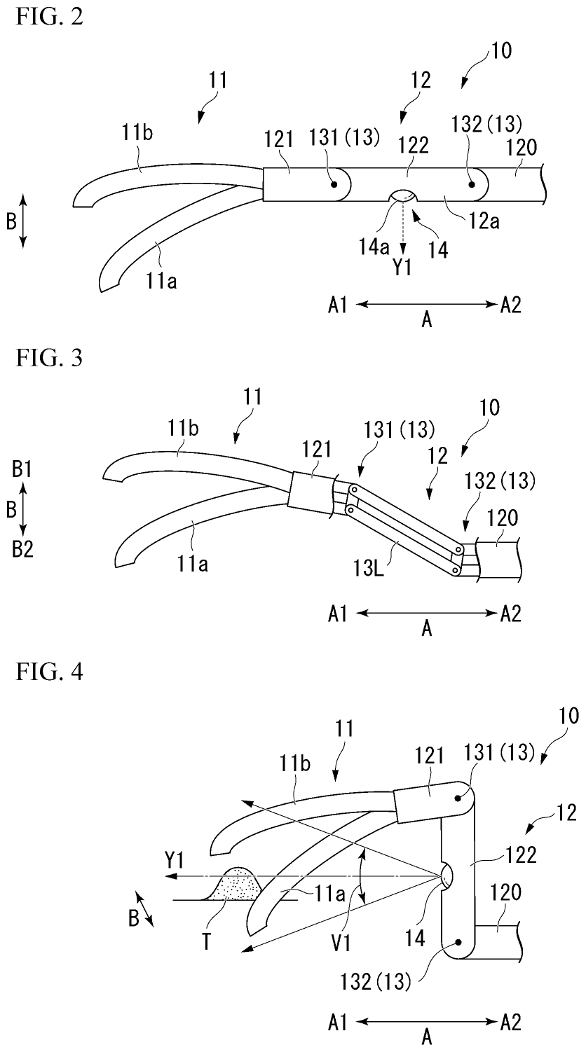

FIG. 2 is a view showing the insertion portion 10 of the treatment device 1.

The insertion portion 10 includes a treatment portion 11 provided on the distal-end side A1, a shaft 12 provided on the proximal-end side A2, and an imaging portion 14 (imaging device) provided on the shaft 12. In the following description, in the longitudinal direction A of the treatment device 1, the side to be inserted into the patient's abdominal cavity is referred to as the “distal-end side A1”, and the operation portion 18 side is referred to as the “proximal-end side A2”.

The treatment portion 11 is a grasping forceps including a first grasping piece 11a and a second grasping piece 11b that can be opened and closed. The first grasping piece 11a and the second grasping piece 11b are opened and closed by an operation wire connected to the operation portion 18. In the following description, the direction in which the first grasping piece 11a and the second grasping piece 11b open and close is referred to as an open-close direction B.

The shaft 12 has a substantially cylindrical shape extending in the longitudinal direction A. The treatment portion 11 is provided on the distal-end side A1 of the shaft 12, and the operation portion 18 is provided on the proximal-end side A2 of the shaft 12. An operation wire for operating the treatment portion 11 is inserted through the shaft 12. The shaft 12 includes a rotation mechanism 13 (pivot point) configured to rotate the distal-end side A1 of the shaft 12 in a direction that intersects the longitudinal direction A. According to the present embodiment, the shaft 12 includes two of rotation mechanism 13. In other words, the shaft 12 includes a first pivot point and a second pivot point.

In the two of rotation mechanism 13, the rotation mechanism 13 provided at the most distal-end side A1 is referred to as a “first rotation mechanism 131”, and the rotation mechanism 13 provided at the proximal-end side A2 than the first rotation mechanism 131 is referred to as a “second rotation mechanism 132”. In addition, the shaft at the proximal-end side A2 of the rotation mechanism 13 (second rotation mechanism 132) that is provided at the most proximal-end side A2 in the shaft 12 is referred to as a “shaft main body 120”, the shaft at the distal-end side A1 of the first rotation mechanism 131 is referred to as a “first segment 121”, and the shaft sandwiched between the first rotation mechanism 131 and the second rotation mechanism 132 is referred to as a “second segment 122”.

The first rotation mechanism 131 connects the first segment 121 and the second segment 122 so as to be rotatable in the open-close direction B. The first segment 121 and the second segment 122 rotate about a rotation axis orthogonal to the longitudinal direction A and the open-close direction B.

The second rotation mechanism 132 connects the second segment 122 and the shaft main body 120 so as to be rotatable in the open-close direction B. The second segment 122 and the shaft main body 120 rotate about a rotation axis orthogonal to the longitudinal direction A and the open-close direction B.

FIG. 3 is a transparent view showing a portion of the shaft 12 on which the rotation mechanism 13 is rotating.

According to the present embodiment, the first rotation mechanism 131 and the second rotation mechanism 132 are connected by a parallel link mechanism 13L. Therefore, when the second segment 122 rotates in one side B1 in the open-close direction B with respect to the shaft main body 120, the first segment 121 correspondingly rotates in the other side B2 in the open-close direction B with respect to the second segment 122. The second segment 122 and the first segment 121 rotate in opposite directions due to the parallel link mechanism. Also, the second segment 122 and the first segment 121 rotate about the axis parallel to the open-close direction B.

As shown in FIG. 2, the imaging portion 14 includes a lens or optical element and an imaging element for photographing the inside of the patient's abdomen. The imaging element may include an imaging sensor or a camera. The imaging portion 14 is housed inside the shaft 12, and includes an observation window 14a formed along an outer circumferential portion 12a of the second segment 122 (a portion being sandwiched by the adjacent rotation mechanisms 13, a portion being at the proximal-end side A2 of the rotation mechanism provided at the most distal-end side A1). The imaging portion 14 is provided on the second segment 122 so as to face an intersecting direction intersecting with the longitudinal direction A. According to the present embodiment, the view direction (center of field angle) Y1 of the imaging portion 14 is a direction orthogonal to the longitudinal direction A of the second segment 122.

Signal lines for transmitting and receiving control signals for controlling the imaging portion 14 and transmission signals for transferring captured images captured by the imaging portion 14 are inserted through the internal space of the shaft 12 or the like to be connected to the control device 3. The control device 3 may transmit and receive the control signals and the transmission signals from the imaging portion 14 of the treatment device 1 by a wireless communication. In this case, it is unnecessary to perform an attachment and a detachment of cables between the treatment device 1 and the control device 3.

The operation portion 18 is provided at the proximal end of the shaft 12 and is a member for operating the treatment portion 11. The operation portion 18 includes a handle 18h. The surgeon can relatively move the handle 18h with respect to other portions of the operation portion 18 to advance and retract the operation wire so as to open and close the treatment portion 11.

### [Endoscope 2]

The endoscope 2 has an elongated and rigid insertion portion 20 that is insertable into the patient's abdominal cavity, and an operation portion 21 provided on the proximal-end side of the insertion portion 20. The insertion portion 20 is introduced into the abdominal cavity through a trocar T punctured in the abdomen B of the patient.

The insertion portion 20 has an imaging portion 22 at the distal end thereof. The imaging portion 22 includes a lens and an imaging element for imaging the inside of the patient's abdomen. The insertion portion 20 introduced into the abdominal cavity is arranged at a position where the imaging portion 22 can image a lesion site as the treatment target in the abdomen. The imaging portion 22 may have an optical zoom or electronic zoom function.

The operation portion 21 is a member operated by the surgeon. The surgeon can change the position and orientation of the imaging portion 22 of the endoscope 2 by moving the endoscope 2 while holding the operation portion 21. The insertion portion 20 may further have a bending portion. By bending the bending portion provided in a part of the insertion portion 20, the position and orientation of the imaging portion 22 can be changed.

Signal lines for transmitting and receiving control signals for controlling the imaging portion 22 and the transmission signals for transferring captured images captured by the imaging portion 22 are wired inside the operation portion 21.

### [Control Device 3]

As shown in FIG. 1, the control device 3 receives the captured images captured by the imaging portion 14 of the treatment device 1 and the imaging portion 22 of the endoscope 2, and transfers the captured images to the display device 4 as display images.

The control device 3 is a program-executable device (computer) including hardware of a processor such as a CPU (Central Processing Unit), a memory and the like. The functions of the control device 3 can be implemented as program (software) functions by the control device 3 reading and executing a program for controlling the processor. At least part of the control device 3 may be configured by a dedicated logic circuit or the like. Furthermore, a similar function can be realized by connecting at least a part of hardware configuring the control device 3 with a communication line.

### [Display Device 4]

The display device 4 is a device configured to display the display images transferred by the control device 3. The display device 4 has a well-known monitor such as an LCD display or the like. The captured image captured by the imaging portion 14 of the treatment device 1 and the captured image captured by the imaging portion 22 of the endoscope 2 are displayed on the monitor. The display device 4 may have a plurality of monitors, and the captured image captured by the imaging portion 14 of the treatment device 1 and the captured image captured by the imaging portion 22 of the endoscope 2 may be displayed on different monitors. Also, the display device 4 may include a head-mounted display or a projector instead of the monitor.

### [Input Device 5]

The input device 5 is a device for the surgeon to input instructions and the like to the control device 3. The input device 5 is configured from either of the well-known devices such as a touch panel, keyboard, mouse, touch pen, foot switch, button or the like, or a combination thereof. The inputs by the input device 5 are transmitted to the control device 3.

### [Operation of Endoscope System 100]

Next, the operations and an operating method of the treatment device 1 and the endoscope system 100 will be described using the laparoscopic surgery as an example.

As shown in FIG. 1, a surgeon or an assistant (hereinafter simply referred to as a “surgeon”) passes the insertion portion 20 of the endoscope 2 through the trocar T punctured in the patient's abdomen B, and introduces the insertion portion 20 into the abdominal cavity. Further, the surgeon passes the insertion portion 10 of the treatment device 1 through the trocar T punctured in the abdomen B of the patient, and introduces the insertion portion 10 into the abdominal cavity.

FIG. 4 is a view for describing the operations of the treatment device 1.

The first rotation mechanism 131 (the rotation mechanism 13 provided on the distal-end side A1 of the imaging portion 14) is configured to operate the first segment 121 to be rotatable in an intersecting direction intersecting to the longitudinal direction A of the second segment 122, that is the direction in which the imaging portion 14 is arranged with respect to the second segment 122. Therefore, the first rotation mechanism 131 is configured to operate the first segment 121 to be rotatable such that at least part of the treatment portion 11 enters the view-filed range V1 of the imaging portion 14.

The imaging portion 14 can observe the treatment portion 11 and the tumor T from a close position, and it is easy to capture the treatment portion 11 and the tumor T in the view-filed range V1 of the imaging portion 14. Furthermore, it is easy for the insertion direction of the treatment device 1 and the view direction (center of field angle) Y1 of the imaging portion 14 to be coincided with each other. Therefore, the surgeon can intuitively treat the tumor T using the treatment portion 11 while observing the captured images of the treatment portion 11 and the tumor T captured by the imaging portion 14. Comparing the situation when the rotation mechanism 13 is rotated and the situation when the rotation mechanism 13 is not rotated, the imaging portion 14 can capture and image the distal end of the treatment portion 11 at the position closer to the center of field angle in the situation when the rotation mechanism 13 is rotated than in the situation when the rotation mechanism 13 is not rotated. As a result, it is possible to make the view-field direction Y1 of the imaging portion 14 and the direction from the proximal end to the distal end of the treatment portion 11 to be substantially coincided with each other. Furthermore, it is possible to prevent a portion of the shaft 12 and the root portion of the treatment portion 14 from being reflected so as to capture and image the distal end of the treatment portion 14 at the center of field angle.

According to the endoscope system 100 including the treatment device 1 according to the present embodiment, it is easy to image the treatment range of the treatment portion 11 by the imaging portion 14 compared to the situation of imaging by the imaging portion 22 of the endoscope 2. By the surgeon observing both of the captured images captured by the imaging portion 14 and the captured images captured by the imaging portion 22 of the endoscope 2, it is possible for the surgeon to perform treatment with a higher accuracy.

As described above, the first embodiment of the present disclosure has been described in detail with reference to the figures, but the specific configuration is not limited to this embodiment, and design changes and the like are included within the scope of the present disclosure. Also, the configurational elements shown in the above-described embodiment and modification examples can be combined as appropriate.

### Modification Example 1

For example, in the above-described embodiment, the first rotation mechanism 131 and the second rotation mechanism 132 are the rotation mechanism 13 that operates correspondingly to the parallel link mechanism 13L; however, the aspect of the rotation mechanism 13 is not limited to the present embodiment. The first rotation mechanism and the second rotation mechanism according to another embodiment may be configurations rotating independently, and may be multi-joint actuators. Also, the rotation axis of the first rotation mechanism and the rotation axis of the second rotation mechanism may be directed to different directions.

### Second Embodiment

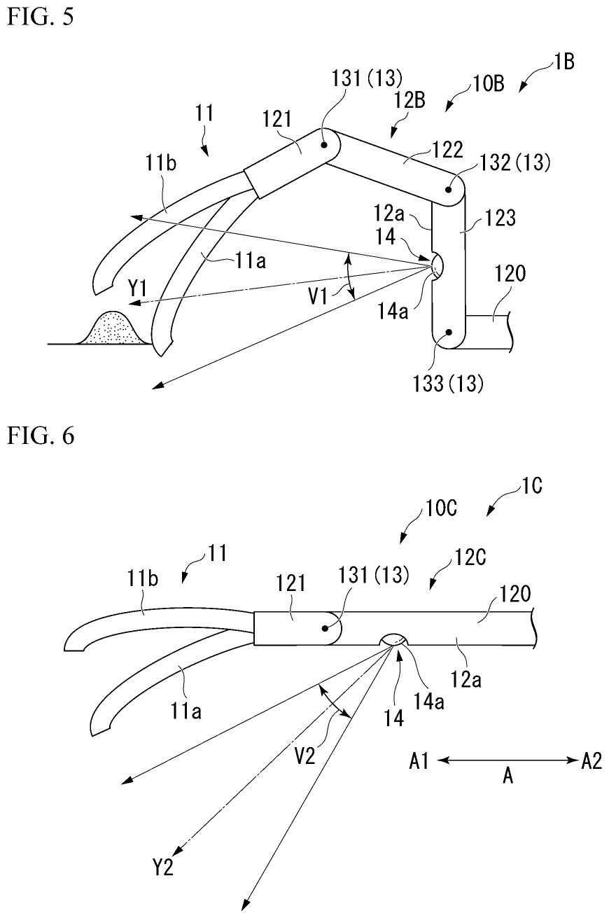

A second embodiment of the present disclosure will be described with reference to FIG. 5. In the following description, the same reference signs are designated to the same configurations as those already described, and the redundant descriptions will be omitted.

FIG. 5 is a view showing an insertion portion 10B of a treatment device 1B according to the present embodiment.

Similar to the treatment device 1 according to the first embodiment, the treatment device 1B is a treatment device connected to the control device 3 and the display device 4. The treatment device 1B has an elongated insertion portion 10B that is insertable into the patient's abdominal cavity, and an operation portion 18. The insertion portion 10B includes a treatment portion 11 provided on the distal-end side A1, a shaft 12B provided on the proximal-end side A2, and an imaging portion 14 provided on the shaft 12B.

The shaft 12B has a substantially cylindrical shape extending in the longitudinal direction A. The treatment portion 11 is provided on the distal-end side A1 of the shaft 12B, and the operation portion 18 is provided on the proximal-end side A2 of the shaft 12B. In the present embodiment, the shaft 12B has three rotating mechanisms 13.

Among the three rotating mechanisms 13, the rotation mechanism 13 provided at the most distal-end side A1 is referred to as a “first rotation mechanism 131”, the rotation mechanism 13 provided at the proximal-end side A2 of the first rotation mechanism 131 is referred to as a “second rotation mechanism 132”, and the rotation mechanism 13 provided at the proximal-end side A2 of the second rotation mechanism 132 is referred to as a “third rotation mechanism 133”. Also, the shaft in the shaft 12 that is provided at the proximal-end side A2 of the rotation mechanism 13 (the third rotation mechanism 133) provided at the most proximal-end side A2 is referred to as a “shaft main body 120”, the shaft at the distal-end side A1 of the first rotation mechanism 131 is referred to as a “first segment 121”, the shaft being sandwiched by the first rotation mechanism 131 and the second rotation mechanism 132 is referred to as a “second segment 122”, and the shaft being sandwiched by the second rotation mechanism 132 and the third rotation mechanism 133 is referred to as a “third segment 123”.

The imaging portion 14 is housed inside the shaft 12B, and includes the observation window 14a formed along the outer circumferential portion 12a of the third segment 123 (the portion being sandwiched by the adjacent rotation mechanisms 13, the portion being at the proximal-end side A2 of the rotation mechanism provided at the most distal-end side A1). The imaging portion 14 is provided to face the intersecting direction intersecting to the longitudinal direction A in the third segment 123. According to the present embodiment, the view direction (center of field angle) Y1 of the imaging portion 14 is orthogonal to the longitudinal direction A of the third segment 123.

The first rotation mechanism 131 and the second rotation mechanism 132 (the rotation mechanism 13 provided on the distal-end side A1 of the imaging portion 14) are configured to operate the first segment 121 and the second segment 122 to be rotatable in the direction in which the imaging portion 14 is arranged with respect to the third segment 123, that is, the intersecting direction intersecting to the longitudinal direction of the third segment 123. Therefore, the first rotation mechanism 131 and the second rotation mechanism 132 are configured to operate the first segment 121 and the second segment 122 to be rotatable such that at least part of the treatment portion 11 enters the view-filed range V1 of the imaging portion 14. Comparing the situation when the rotation mechanism 13 is rotated and the situation when the rotation mechanism 13 is not rotated, the imaging portion 14 can capture and image the distal end of the treatment portion 11 at the position closer to the center of field angle in the situation when the rotation mechanism 13 is rotated than in the situation when the rotation mechanism 13 is not rotated.

According to the treatment device 1B according to the present embodiment, similar to the treatment device 1 according to the first embodiment, it is easy for the imaging portion 14 to image the treatment range by the treatment portion 11 compared to the situation in which the imaging portion 22 of the endoscope 2 images.

As described above, the second embodiment of the present disclosure has been described in detail with reference to the figures; however, the specific configuration is not limited to the present embodiment, and design changes and the like are included within the scope of the present disclosure. Also, the configurational elements shown in the above-described embodiment and modification example can be combined as appropriate.

### Third Embodiment

A third embodiment according to the present disclosure will be described with reference to FIG. 6 to FIG. 7. In the following description, the same reference signs are designated to the same configurations as those already described, and redundant descriptions will be omitted.

FIG. 6 is a view showing an insertion portion 10C of a treatment device 1C according to the present embodiment.

The treatment device 1C is a treatment device connected to the control device 3 and the display device 4, similar to the treatment device 1 according to the first embodiment. The treatment device 1C includes an elongated insertion portion 10C that is insertable into the patient's abdominal cavity, and an operation portion 18. The insertion portion 10C includes the treatment portion 11 provided on the distal-end side A1, a shaft 12C provided on the proximal-end side A2, and the imaging portion 14 provided in the shaft 12C.

The shaft 12C has a substantially cylindrical shape extending in the longitudinal direction A. The treatment portion 11 is provided on the distal-end side A1 of the shaft 12C, and the operation portion 18 is provided on the proximal-end side A2 of the shaft 12C. According to the present embodiment, the shaft 12C includes one rotation mechanism 13.

The one rotation mechanism 13 having the shaft 12C is referred to as the “first rotation mechanism 131”. Also, the shaft at the proximal-end side A2 of the rotation mechanism 13 (first rotation mechanism 131) that is provided at the most proximal-end side A2 in the shaft 12C is referred to as the “shaft main body 120”, and the shaft at the distal-end side A1 of the first rotation mechanism 131 is referred to as the “first segment 121”.

The imaging portion 14 is housed inside the shaft 12C, and includes an observation window 14a that is formed along the outer circumferential portion 12a of the shaft main body 120 (the portion at the proximal-end side A2 of the rotation mechanism 13 provided at the most distal-end side A1). The imaging portion 14 is provided in the shaft main body 120 to be directed to the intersecting direction intersecting to the longitudinal direction A. According to the present embodiment, the view direction (center of field angle) Y2 of the imaging portion 14 is the intersecting direction intersecting to the longitudinal direction A of the shaft main body 120, that is, the direction toward the distal-end side A1 of the shaft main body 120.

FIG. 7 is a view showing the shaft 12C in which the rotation mechanism 13 is rotating.

The first rotation mechanism 131 (the rotation mechanism 13 provided on the distal-end side A1 of the imaging portion 14) is configured to operate the first segment 121 to be rotatable in the direction where the imaging portion 14 is arranged with respect to the shaft main body 120, that is, the intersecting direction intersecting to the longitudinal direction of the shaft main body 120. Therefore, the first rotation mechanism 131 is configured to operate the first segment 121 to be rotatable such that at least part of the treatment portion 11 enters the view-field range V2 of the imaging portion 14. Comparing the situation when the rotation mechanism 13 is rotated and the situation when the rotation mechanism 13 is not rotated, the imaging portion 14 can capture and image the distal end of the treatment portion 11 at the position closer to the center of field angle in the situation when the rotation mechanism 13 is rotated than in the situation when the rotation mechanism 13 is not rotated.

According to the treatment device 1C according to the present embodiment, similar to the treatment device 1 according to the first embodiment, it is easy for the imaging portion 14 to image the treatment target range by the treatment portion 11 compared to the case in which the imaging portion 22 of the endoscope 2 images.

As described above, the third embodiment according to the present disclosure has been described in detail with reference to the figures; however, the specific configuration is not limited to this embodiment, and design changes and the like are included within the scope of the present disclosure. Also, the configurational elements shown in the above-described embodiment and modification examples can be combined as appropriate.

### Fourth Embodiment

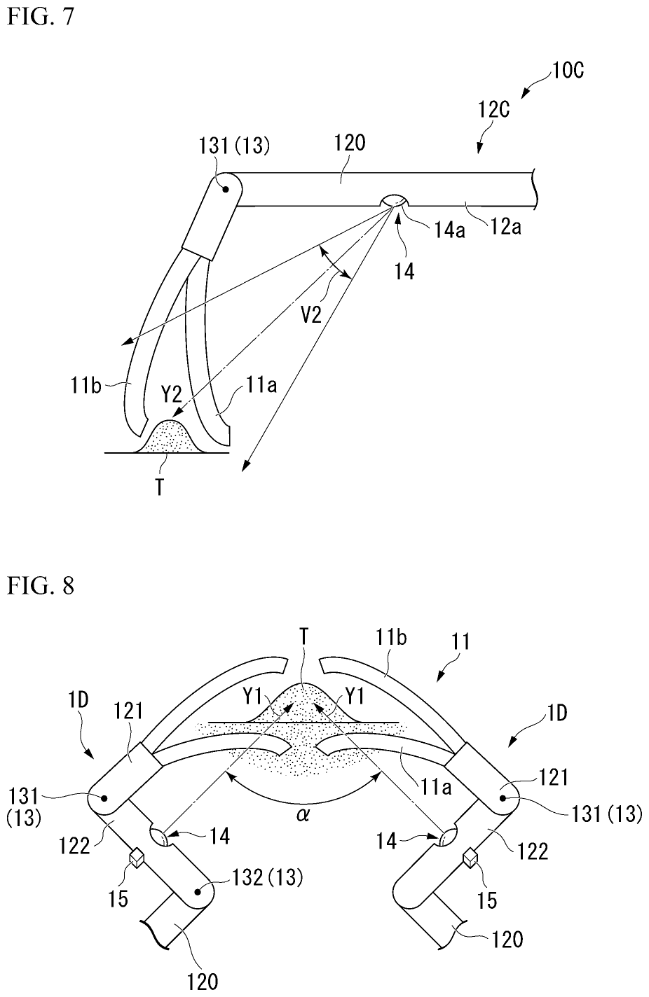

A fourth embodiment according to the present disclosure will be described with reference to FIG. 8. In the following description, the same reference signs are designated to the same configurations as those already described, and redundant descriptions will be omitted.

FIG. 8 is a view showing part of an endoscope system 100D according to the present embodiment.

The endoscope system 100D includes two treatment devices 1D, the endoscope 2, the control device 3, the display device 4, and the input device 5. The endoscope system 100D is a system that performs procedures in the laparoscopic surgery by inserting the treatment device 1D, the endoscope 2, and the like through different holes (openings) opened in the abdominal wall.

The treatment device 1D is different from the treatment device 1 according to the first embodiment only in that the treatment device 1D includes an orientation sensor 15. The orientation sensor 15 is provided at a portion of the second segment 122 that is close to the imaging portion 14. The orientation data detected by the orientation sensor 15 is acquired by the control device 3. The control device 3 calculates the view direction Y1 and the like of the imaging portion 14 based on the orientation data.

The control device 3 receives the captured images captured by the imaging portions 14 of the two treatment devices 1D, generates the display images and transfers the display images to the display device 4. The control device 3 may combine the captured images captured by the imaging portions 14 of the two treatment devices 1 to generate the display image (two-dimensional image) with an enlarged view field range. Also, the control device 3 may generate a three-dimensional image capable of realizing the three-dimensional stereoscopic viewing from the captured images captured by the imaging portions 14 of the two treatment devices 1 based on the orientation data detected by the orientation sensor 15. In the case in which the control device 3 generates the three-dimensional images, the display device 4 may be a three-dimensional monitor capable of displaying the three-dimensional images.

The control device 3 may control the rotation mechanism 13 of each treatment device 1D such that an angle (the convergence angle in the stereoscopic vision) α formed by the view directions (center of field angles) Y1 of the imaging portions 14 of the two treatment devices 1D becomes an appropriate angle. Also, in a case in which the angle α is excessively large, since the stereoscopic vision becomes difficult, the control device 3 may switch the generated display image from the three-dimensional image to the two-dimensional image. In this case, it is possible to enlarge the view filed of the two-dimensional image by pasting together the images captured by the imaging portions 14 of the two treatment devices 1D.

As described above, the fourth embodiment according to the present disclosure has been described in detail with reference to the figures; however, the specific configuration is not limited to the present embodiment, and design changes and the like are included within the scope of the present disclosure. Also, the configuration elements shown in the above-described embodiment and modification examples can be combined as appropriate.

### Modification Example

For example, in the above embodiment, the treatment portion 11 is a grasping forceps; however, the aspect of the treatment portion 11 is not limited to this configuration. Another aspect of the treatment portion may be an energy device such as a high frequency knife and the like.

### Modification Example

For example, in the above embodiment, the shaft is configured to have one to three rotation mechanisms 13; however, the aspect of the shaft is not limited to this configuration. Another aspect of the shaft may have four or more rotation mechanisms 13, or may have different types of rotation mechanisms.

### INDUSTRIAL APPLICABILITY

The present disclosure can be applied to an endoscope system that performs a treatment using a treatment device and an endoscope.

### REFERENCE SIGNS LIST

100, 100D endoscope system
1, 1B, 1C, 1D treatment device (treatment device)
10, 10B, 10C insertion portion
11 treatment portion
12, 12B, 12C shaft
12a outer circumferential portion
120 shaft main body
121 first segment (rotation shaft)
122 second segment (rotation shaft)
123 third segment
13 rotation mechanism
131 first rotation mechanism (rotation mechanism)
132 second rotation mechanism (rotation mechanism)
133 third rotation mechanism (rotation mechanism)
14 imaging portion
14a observation window
15 orientation sensor
18 operation portion
3 control device
4 display device
5 input device
Y1, Y2 view direction, center of view angle
V1, V2 view-filed range

What is claimed is:

1. A treatment device, comprising:
a shaft extending along a longitudinal axis and including a plurality of segments arranged along the longitudinal axis, wherein a first segment of the plurality of segments is adjacent to a second segment of the plurality of segments;
a treatment portion at a distal-end side of the shaft;
an operation portion at a proximal-end side of the shaft and configured to operate the treatment portion; and
an imaging device,
wherein, along the longitudinal axis of the shaft, the first segment is at the distal-end side of the second segment,
wherein the imaging device is provided in the second segment,
wherein the first segment is rotatably attached to the second segment at a first pivot point, and
wherein the first segment is rotatable about the first pivot point relative to the second segment such that a longitudinal axis of the first segment intersects a longitudinal axis of the second segment at a first angle that is less than 180 degrees and at least a part of the treatment portion enters a field of view of the imaging device.

2. The treatment device according to claim 1, wherein a direction of a centerline of the field of view of the imaging device is orthogonal to the longitudinal axis of the second segment.

3. The treatment device according to claim 1, wherein a centerline of the field of view of the imaging device is oriented toward the distal-end side of the shaft.

4. The treatment device according to claim 1, wherein the plurality of segments includes a third segment,
wherein the third segment is rotatably attached to the second segment at a second pivot point, and
wherein the second segment is rotatable about the second pivot point relative to the third segment such that the longitudinal axis of the second segment intersects a longitudinal axis of the third segment at a second angle that is less than 180 degrees.

5. The treatment device according to claim 4, wherein a direction of a centerline of the field of view of the imaging device is orthogonal to the longitudinal axis of the second segment.

6. The treatment device according to claim 1, wherein the imaging device is housed inside the second segment, and wherein the imaging device includes an observation window formed along a portion of an outer surface of the second segment.

7. The treatment device according to claim 1, wherein the shaft has a shape that is configured to be insertable into a trocar.

8. The treatment device according to claim 1, wherein the treatment portion includes a grasping forceps or an energy device, wherein the operation portion includes a handle, a knob, or a joystick, and wherein the imaging device includes an imaging sensor, a camera, a lens or an optical element.

9. An endoscope system, comprising:
a treatment device according to claim 1;
an endoscope; and
a processor configured to process a first image captured by the imaging device of the treatment device and a second image captured by the endoscope.

10. An endoscope system, comprising:
a plurality of treatment devices according to claim 1, including a first treatment device and a second treatment device;
an endoscope; and
a processor configured to generate a three-dimensional image based on images captured by imaging devices of the plurality of treatment devices.

11. A treatment device, comprising:
a first shaft portion extending along a longitudinal axis;
a second shaft portion rotatably attached to the first shaft portion, the second shaft portion configured to rotate in a direction intersecting with the longitudinal axis;
a treatment portion provided at a distal end of the second shaft portion;
an operation portion provided at a proximal end of the first shaft portion and configured to operate the treatment portion; and
an imaging device provided on the first shaft portion such that at least part of the treatment portion enters a field of view of the imaging device when the second shaft portion is rotated.

12. The treatment device according to claim 11, wherein a direction of a centerline of the field of view of the imaging device is orthogonal to the longitudinal axis of the first shaft portion.

13. The treatment device according to claim 11, wherein a centerline of the field of view of the imaging device is oriented toward the distal-end side of the second shaft portion.

14. The treatment device according to claim 11, wherein the second shaft portion is configured to rotate toward the imaging device.

15. The treatment device according to claim 14, wherein the first shaft portion comprises a distal end shaft portion and a proximal end shaft portion, and
wherein the distal end shaft portion is rotatably attached to the proximal end shaft portion.

16. The treatment device according to claim 15, wherein the distal end shaft portion includes the imaging device.

17. The treatment device according to claim 11, wherein the imaging device is housed inside the first shaft portion, and wherein the imaging device includes an observation window formed along a portion of an outer surface of the first shaft portion.

18. The treatment device according to claim 11, wherein the first shaft portion has a shape that is configured to be inserted into a trocar.

19. The treatment device according to claim 11, wherein the treatment portion includes a grasping forceps or an energy device, wherein the operation portion includes a handle, a knob, or a joystick, and wherein the imaging device includes an imaging sensor, a camera, a lens or an optical element.

20. An endoscope system, comprising:
a treatment device according to claim 11;
an endoscope; and
a processor configured to process a first image captured by the imaging device of the treatment device and a second image captured by the endoscope.
